(19) United States
(12) Patent Application Publication
(10) Pub. No.: US 2004/0138573 A1
(43) Pub. Date: Jul. 15, 2004

(54) Mechanism of heartbeat monitor

(76) Inventor: Shui-Jung Chen, San Chung Taipei (TW)

Correspondence Address: Leong C. Lei, PMB#1008, 1867 Ygnacio Valley Road, Walnut Creek, CA, 94598-3214, US

(21) Appl. No.: 10/342,384

(22) Filed: Jan. 15, 2003

(51) Int. Cl. A61B 5/0402

(52) U.S. Cl. 600/509

(57) ABSTRACT

An improvement on mechanism of heartbeat monitor which primarily comprises a shell, a circuit board, a conductible section, and a strap; said heartbeat monitor detects the heartbeat count while worn on the breast of people and wirelessly transmits the detected heartbeat counts. The present invention is characterized in: said shell comprising an upper cover, a bottom cover, and a battery cover; two conductible portions respectively combining two sides of upper cover; one end of a conductible rubber sheet being deeply inserted into the interior of upper cover; at the bottom surface of the circuit board, two metal contact portions being installed at a location where conductible rubber sheet being exposed to the interior of upper cover so as to make the contact portion directly touch the conductible rubber sheet to reach electrical connection while the circuit board being assembled inside the upper cover; a bottom cover, and a battery cover being assembled to accomplish assembling procedure.

### BACKGROUND OF THE INVENTION

1. Field of the Present Invention

The present invention relates to an improvement on mechanism of a heartbeat monitor, particularly it relates to a mechanism of heartbeat monitor worn on the breast of people. The heartbeat monitor of the present invention has a contact portion of which the conductible rubber sheet is directly and conveniently accomplished with electrical connection while assembled; the heartbeat monitor of the present invention also has a mechanism for changing the battery easily.

2. Description of the Prior Art

The mechanism of prior heartbeat monitor as shown in FIG. 5 is an assembly section drawing of U.S. Pat. No. 5,464,021, of which the assembly procedure is: first, holding a conductible rubber sheet (b) respectively at two sides of a circuit board (a) to accomplish electrical connection; then, molding said circuit board (a) and conductible rubber sheet (b), which have been connected, to have an upper shell (c) wrapping said circuit board (a) and conductible rubber sheet (b) as one body; covering with a bottom cover (not shown in the drawing) to accomplish the assembling procedure of the product. To use the product, installing a battery into said circuit board and tying a strap (not shown in the drawing) to the side of outer shell which extends from the upper shell (c) to both sides and wraps said conductible rubber sheet (b), finally wearing it on the breast of people.

The major defect of mechanism of prior heartbeat monitor is that during its assembling procedure, it is necessary to mold the connected circuit board (a) and conductible rubber sheet (b) with special machine, which causes that the structure of circuit board (a) and conductible rubber sheet (b) wrapped and fixed inside the upper shell (c) material, so it is not easy to separate them after being integrally molded; therefore, while a defect such as electrical connection was found between circuit board (a) and conductible rubber sheet (b) during circuit testing, it is impossible to read and adjust or repair but to dispose; this consequently increase the cost. Moreover, as shown in the prior structure, wherein the bottom cover is normally a single member, which causes that the user needs to disassemble the bottom cover completely in order to change the battery while using it; therefore, it is not convenient to disassemble and therefore to damage the circuit board frequently.

In view of this, the inventor of the present invention thus dedicates himself to the research and development of the improved mechanism of heartbeat monitor.

### SUMMARY OF THE INVENTION

The primary purpose of the present invention is to provide an improved mechanism of heartbeat monitor of which the parts are so simple that it is convenient and quick to assemble, which effectively brings the cost down.

The secondary purpose of the present invention is to provide an improved mechanism of heartbeat monitor of which a battery cover is installed in the bottom cover, which makes the user easy to change the battery and decreases the number of disassembling the bottom cover in order to prevent the circuit board from being broken.

To reach the aforementioned purpose, this invention provides a shell comprising an upper cover, a bottom cover, and a battery cover; a conductible portion has its bottom surface installed a conductible rubber sheet; two conductible portions respectively combining two sides of upper cover; one end of a conductible rubber sheet being deeply inserted into the interior of upper cover; at the bottom surface of the circuit board, two metal contact portions being installed at a area where conductible rubber sheet being exposed to the interior of upper cover so as to make the contact portion directly touch the conductive rubber sheet for electrical connection while the circuit board being assemble, inside the upper cover; a bottom cover, and a battery cover being assembled to accomplish assembling procedure.

Therefore, each part of the present invention is independently pre-assembled, then is quickly assembled with screws; moreover, the malfunctioning part is replaced or repaired during testing after assembling in order to reach the preset purpose of the present invention. In addition, the bottom cover of the present invention is specifically separated from battery cover as two members, which contributes to the advantage of solely opening the battery cover for user to change battery and refrains from damaging the circuit board by accident.

In order to let the receiver more clearly understand the mechanism, and features of the present invention, a preferred embodiment is described in detail as follows coordinating with drawings, which is given to illustrate characteristics of the present invention but not to limit the invention.

### BRIEF DESCRIPTION OF THE DRAWINGS

FIG. 1 is a perspective view of the present invention.

FIG. 2 is a bottom view of explosion drawing of the present invention.

FIG. 3 is a schematic drawing of the preferred embodiment of the present invention.

FIG. 4 is a section view of the present invention.

FIG. 5 is a section view of prior heartbeat monitor.

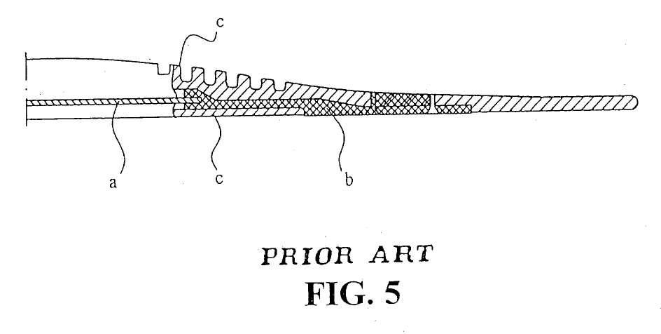

### DETAILED DESCRIPTION OF THE PREFERRED EMBODIMENT

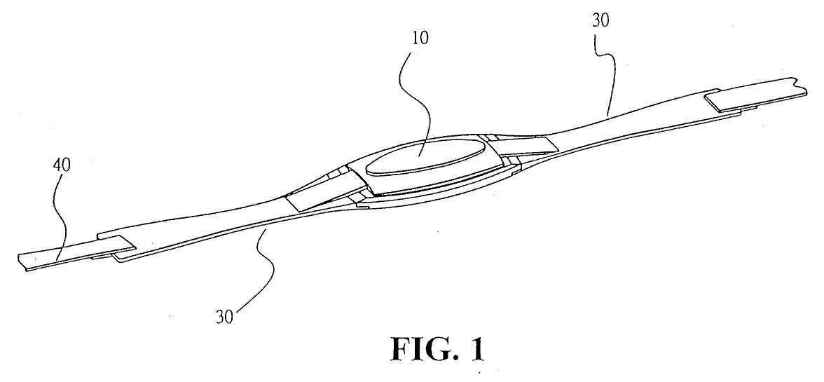

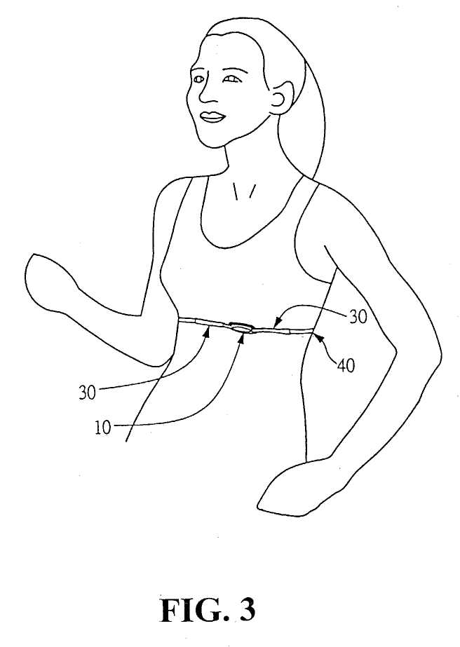

Referring to FIG. 1 to FIG. 3, the improved mechanism of heartbeat monitor of the present invention comprises a shell 10, a circuit board 20, a conductive portion 30, and a wearing strap 40; the improved mechanism of heartbeat monitor worn on people's breast in order to monitor heartbeat counts and to wirelessly transmit the monitored heartbeat counts. The shell 10 comprises an upper cover 11, a bottom cover 12, and a battery cover 13. The upper cover 11 at least has its two sides each installed an insert hole 14 to combine the conductive portion, and the bottom cover 12 is fixed to the bottom surface of the upper cover 11 with screw, and a rubber ring 16 is installed between the bottom cover 12 and the circuit board 20 to increase its ability of damp proof.

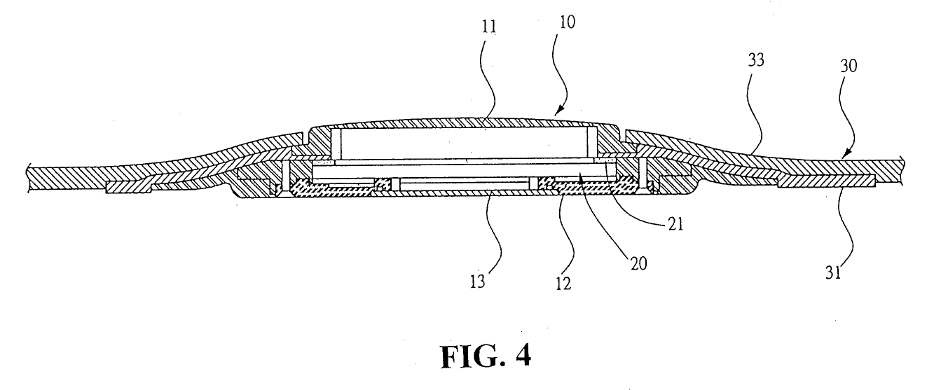

The circuit board 20 monitors the heartbeat of user and wirelessly transmits the monitored heartbeat counts; both sides of the bottom surface of the circuit board 20 each has a contact portion 21 respectively. In this preferred embodiment, the contact portion 21 is integrally fixed to the electrical touching point located at the bottom surface of said circuit board with a metal plate so as to make the contact portion 21 protrude downward to the bottom surface of said circuit board 20 (as shown in FIG. 4).

The conductive portion 30 is a strap body connecting and fixing to both sides of the upper cover. The conductive portion 30 comprises a rubber body 33 and a conductible rubber sheet 31. Said conductive rubber sheet 31 is at one end which connects the shell and is at the bottom of said rubber body 33 where a portion of said conductive sheet exposes to the exterior of said rubber body; and there is a hole 32 at the other end of said rubber body 33.

The wearing elastic strap 40 connects a hole 32 at the other end (external side) of said conductive portion 30. The wearing elastic strap is made of a elastic material, which is convenient to be worn on user's breast.

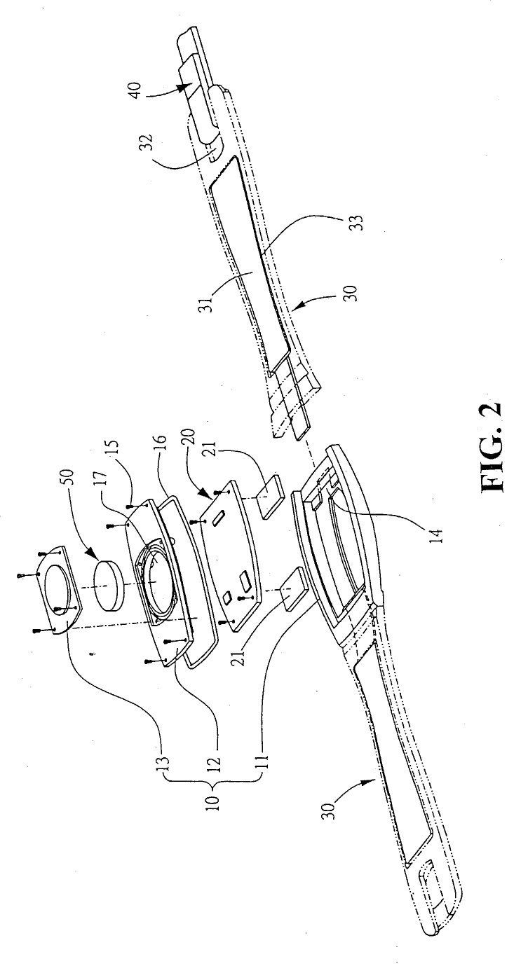

While the aforementioned mechanism is assembled (referring to FIG. 2 and FIG. 4), the preferred embodiment has an insert hole 14 at both sides of the upper cover respectively; one end of the conductible rubber sheet 31 of the conductive portion 30 is inserted into said insert hole 14 so as to make the conductive rubber of that end expose to the accommodating room of the upper cover 11 (that end of the conductible rubber sheet is temporarily fixed by glue or double-sided tape), which is then molded to make the rubber body 33 of the conductive portion 30 melted to combine the upper cover as an integral body; then, a circuit board 20 is installed on said integral body in a way that the contact portion 21 at the bottom surface of the circuit board directly presses the conductive rubber sheet 31 exposed in the accommodating room of the upper cover 11 in order to reach the goal of quickly assembling and electrically connecting; finally, the bottom cover 12 and the battery cover 13 are assembled to accomplish the overall assembling work. As mentioned before, the manufacturing and assembling of the present invention is simpler and quicker than those of the prior art; moreover, in case that any fault is found during test after assembling, it is very easy for the present invention for parts repairing replacement. So, the present invention is more useful and more economically valuable.

In addition, shown as aforementioned mechanism, the bottom cover 12 of the present invention is installed with an opening 17, which is a little bigger than the battery, at the location corresponding to the circuit board 20 where a battery is installed; and a battery cover 13 is used to seal in order to become a two-piece member with a rubber ring therebetween for sealing; so that while the user wants to change the battery (not shown in the drawing), he needs merely to open the battery cover 13 to quickly change the battery so as to prevent the circuit board from being damaged by accident while opening the bottom cover which is required by the prior art.

To sum up, an improved mechanism of heartbeat monitor of the present invention actually reaches the anticipated effect and goal, which is industrially valuable; so, the present invention perfectly fulfills the requirement of the award of an invention patent.

I claim:

1. An improved mechanism of heartbeat monitor, comprising: a shell, a circuit board, a conductible portion, and a elastic strap; said heartbeat monitor is worn on people's breast in order to monitor heartbeat count wirelessly transmitted by said heartbeat monitor; said heartbeat monitor is characterized in: said shell comprising an upper cover, a bottom cover, and a battery cover; two conductible portions respectively combining two sides of upper cover, wherein one end of a conductible rubber sheet being deeply inserted into the interior of upper cover and exposed; at the bottom surface of the circuit board, two contact portions being installed at the location where conductible rubber sheet being exposed to the interior of upper cover so that while said circuit board being installed inside said upper cover, the contact portion directly pressing the surface of the conductible rubber sheet in order to perfectly contact and quickly assemble.

2. An improved mechanism of heartbeat monitor as defined in claim 1, wherein each side of the upper cover has an insert hole; one end of the conductible rubber of the conductible portion is inserted into said insert hole so as to make the conductible rubber of that end expose to the accommodating room of the upper cover; further, the rubber body of the conductible portion and the upper cover are integrally molded into one body.

3. An improved mechanism of heartbeat monitor as defined in claim 1, wherein the contact portion at the bottom surface of the circuit board is integrally fixed to the electrical touching point by a metal plate so as to make the contact portion protrude downward to the bottom surface of said circuit board.

4. An improved mechanism of heartbeat monitor as defined in claim 1, wherein a sealing rubber ring is installed between the upper cover and the bottom cover.
